(19) United States
(12) Patent Application Publication
(10) Pub. No.: US 2007/0118007 A1
(43) Pub. Date: May 24, 2007

(54) INTERNAL OLEFINS PROCESS

(76) Inventors: Howard Lam-Ho Fong, Sugar Land, TX (US); Brendan Dermot Murray, Houston, TX (US)

Correspondence Address: SHELL OIL COMPANY, P O BOX 2463, HOUSTON, TX, 772522463, US

(21) Appl. No.: 11/553,365

(22) Filed: Oct. 26, 2006

Related U.S. Application Data
(60) Provisional application No. 60/731,174, filed on Oct. 28, 2005.

(51) Int. Cl. C07C 2/04; C07C 2/34

(52) U.S. Cl. 585/511; 585/510

(57) ABSTRACT

This invention provides a process for making internal olefins which comprises isomerizing a feed comprising one or more internal olefin(s) in the presence of an isomerization catalyst to produce alpha olefins and reacting said alpha olefins in the presence of a dimerization catalyst to produce internal olefins.

### CROSS REFERENCE TO RELATED APPLICATION

This application claims priority to U.S. Provisional Application Ser. No. 60/731,174 filed Oct. 28, 2005, the entire disclosure of which is herein incorporated by reference.

### FIELD OF THE INVENTION

The present invention relates to a process for converting lower carbon number internal olefins into higher carbon number internal olefins.

### BACKGROUND OF THE INVENTION

Branched internal olefins have been made by structural or skeletal isomerization of linear olefins to their corresponding methyl branched isoolefins. U.S. Pat. No. 5,510,306 describes one such process. Internal olefins have been made by dimerization of linear alpha olefins with a variety of dimerization catalysts.

In many commercial operations, lower carbon number internal olefins are produced. It would be advantageous to have a process which would convert these lower carbon number internal olefins, which are of low value, into higher carbon number internal olefins, preferably with some branching, which have a higher value and may be converted into the type of alcohols which may be used to make detergent products. The present invention provides such a process.

U.S. Pat. No. 6,291,733 describes a process for dimerizing alpha olefins to produce mostly linear internal olefins. This reaction is said to be highly selective. Internal olefins do not react by this dimerization process.

### SUMMARY OF THE INVENTION

This invention provides a process for making internal olefins which comprises isomerizing a feed comprising one or more internal olefin(s) in the presence of an isomerization catalyst to produce alpha olefin(s), and reacting said alpha olefins in the presence of a dimerization catalyst to produce internal olefin(s) which have a higher carbon number than the feed internal olefin(s).

### DETAILED DESCRIPTION OF THE INVENTION

The product internal olefins may have a higher carbon number than the feed internal olefins and may be C₆₋₄₀, C₈₋₂₀, or C₁₂₋₁₈ linear and/or alkyl-branched internal olefins. The feed internal olefin(s) may have a lower carbon number than the product internal olefins and may be C₄₋₂₄, C₄₋₂₀, C₄₋₁₄, C₄₋₁₂, C₄₋₁₀, or C₄₋₈ internal olefins. The feed internal olefin stream may optionally contain one or more alpha olefin(s).

The isomerization in this process may be carried out in a different manner than isomerization is usually carried out. It is well understood that internal olefins may be reacted with an isomerization catalyst under isomerization conditions to produce alpha olefins (double bond isomerization).

It is also well understood that it is difficult to make alpha olefins in high conversion from internal olefins solely by an isomerization reaction. The reaction is an equilibrium reaction which favors the presence of internal olefins. In the present invention, the reaction produces alpha olefins from the starting feed of internal olefins. The alpha olefins are removed from the reaction mixture by dimerization to internal olefins and are replenished by the equilibrium of the isomerization reaction. The process of the invention may be carried out under conditions wherein the amount of alpha olefin(s) produced may be as high as possible, preferably the equilibrium amount of the alpha olefins in the isomerization reaction mixture or as close to the equilibrium amount as possible

Contacting the alpha olefins with the dimerization catalyst allows the dimerization reaction of the alpha olefins to proceed to produce longer chain internal olefins (than the feed internal olefins) from the alpha olefins produced during the isomerization reaction. It is preferred that the dimerization and isomerization catalysts be compatible with each other so as not to react such that the activity is reduced. Preferably, both catalysts should either be basic or acidic. For example, a homogeneous solution of a basic catalyst should not generally be mixed with a soluble acid catalyst. There are engineering solutions to enable the use of a solid acid and a solid base as long as they don't contact each other.

The isomerization conditions used herein may be chosen from a wide variety of catalysts and isomerization processes. Some of these processes include those described in U.S. Pat. Nos. 3,786,112, 4,749,819, 4,727,203, 5,107,047, 5,177,281, and 5,510,306, the disclosures of which are all herein incorporated by reference in their entirety. In instances that the isomerization reaction is separated from the reaction involving the dimerization catalyst, the conditions may include operating at a temperature of from about 0 to about 500° C., a pressure from about 1 to about 10,000 kPa, and, in a continuous process, a weight hourly space velocity of from about 0.1 to about 100. Generally, temperatures of about 200° C. or less may be sufficient and pressures of from about atmospheric to about 5000 kPa may be used. The thermodynamic equilibrium concentration of α-olefins in an olefin mixture of the same carbon number increases as the temperature increases in the range of 0 to 500° C. The temperature may be as high as possible to maximize the amount of alpha olefins produced. However, the temperature should not be high enough to decompose the dimerization catalyst and/or the isomerization catalyst.

Almost any isomerization catalyst may be used but it is preferred that it be compatible with the dimerization catalyst chosen. Among the isomerization catalysts that may be used are the catalysts which are disclosed in U.S. Pat. Nos. 3,786,112, 4,749,819, 4,727,203, 5,107,047 5,177,281, and 5,510,306, which are incorporated by reference.

Suitable isomerization catalysts for use in this invention include catalysts comprising Group VIII noble metals, i.e., palladium, platinum, or ruthenium; niobium, or vanadium oxides; Group I, Group II, or Group III metal oxides including sodium oxide, potassium oxide, magnesium oxide, calcium oxide, zinc oxide, gamma-alumina, bauxite, eta-alumina, barium oxide, strontium oxide and mixtures thereof; and Group I metal carbonates on alumina.

Other isomerization catalysts which may be used include alumino silicate catalysts. A preferred alumino silicate catalyst is a ferrierite alumino silicate catalyst defined as having eight and ten member ring channels. Other preferred alumino silicates are ferrierite catalysts which are exemplified by the ZSM-35 alumino silicate described in U.S. Pat. No. 4,016,245, the disclosure of which is incorporated herein by reference in its entirety, or by a piperidine derived ferrierite as described in U.S. Pat. No. 4,251,499, the disclosure of which is herein incorporated by reference in its entirety. Other useful zeolites include Theta-1, ZSM-12, ZSM-22, ZSM-23, and ZSM-48. These alumino silicates may be associated with a catalytic metal, preferably selected from Group VIII or Group VIB of the periodic table. These metals may be exemplified by palladium, platinum, ruthenium, nickel, cobalt, molybdenum, osmium, and may be present in combination with one another. These catalytic metals may be present in quantities from about 0.1 weight percent to about 25 weight percent of the total catalyst composition.

The ZSM-22 catalyst is more particularly described in U.S. Pat. No. 4,556,477, the entire contents of which are herein incorporated by reference. The ZSM-23 catalyst is more particularly described in U.S. Pat. No. 4,076,842, the entire contents of which are herein incorporated by reference.

The MCM-22 catalyst described in U.S. Pat. No. 5,107,047 may also be used as the isomerization catalyst in the present invention. Zeolite MCM-22 may have a composition involving the molar relationship:
X₂O₃:(n)YO₂
wherein X is a trivalent element, such as aluminum, boron, iron and/or gallium, preferably aluminum, Y is a tetravalent element such as silicon and/or germanium, preferably silicon, and n is at least about 10, usually from about 10 to about 150, more usually from about 10 to about 60, and even more usually from about 20 to about 40. In the as synthesized form, zeolite MCM-22 may have a formula, on an anhydrous basis and in terms of moles of X₂O₃ oxides per n moles of YO₂ oxides, as follows:
(0.005-0.1)Na₂O:(1-4)R:X₂O₃:nYO₂
wherein R is an organic component. The Na₂O and R components are associated with the zeolite as a result of their presence during crystallization, and are easily removed by post-crystallization methods. This zeolite, especially in its metal, hydrogen, and ammonium forms, can be beneficially converted to another form by thermal treatment.

In another embodiment, an alkali metal catalyst, preferably a sodium/potassium (NaK) catalyst, is used as discussed in U.S. Pat. No. 4,749,819, which is herein incorporated by reference in its entirety. The preferred NaK catalyst is a eutectic mixture of sodium and potassium that is put on an alumina or silica support. A NaK catalyst may be made according to the teachings of U.S. Pat. No. 3,405,196, which is herein incorporated by reference in its entirety, by using a mixture of sodium and potassium as the alkali metal component.

As described above, the internal olefin feed may optionally contain some α-olefins. In some embodiments of this invention, it may be preferred that α-olefins be present in the feed. In one such embodiment, the α-olefins may be ethylene, propylene, or a mixture thereof. The presence of these α-olefins will allow the production of internal olefins having 6 or 7 carbon atoms.

In instances that the isomerization reaction is separated from the reaction involving the dimerization catalyst, the reaction involving the dimerization catalyst may be operated at temperatures up to about 200° C., preferably from about −10 to about 100° C., and more preferably from about 10 to 50° C. The pressure may range from about 1 to about 10,000 kPa, preferably from atmospheric pressure to about 5000 kPa.

There are a variety of dimerization catalysts which may be used in the present invention. These catalysts include those described in U.S. Pat. Nos. 4,252,987, 4,859,646, 6,222,077, 6,291,733, and 6,518,473, all of which are herein incorporated by reference. One such catalyst may comprise a dicyclopentadienyl halogenated titanium compound, an alkyl aluminum halide, and a nitrogen Lewis phase. Other such catalysts may include 1) a palladium compound, 2) a chelate ligand comprising a compound containing at least 2 nitrogen atoms which are connected through a chain comprising two or more carbon atoms, 3) a protonic acid, and 4) a salt of copper, iron, zinc, tin, manganese, vanadium, aluminum, or a group VIB metal. In another embodiment, the catalyst may be one wherein a metal, preferably nickel, is bound to at least one hydrocarbyl group or a catalyst which consists of complexes formed by admixing at least one nickel compound with at least one alkyl aluminum compound and optionally a ligand. The catalyst may also be a catalyst comprising a combination of a nickel carboxylate or a nickel chelate with an alkyl aluminum halide or an alkyl aluminum alkoxide. Furthermore, catalysts for dimerization may be virtually any acidic material including zeolites, clays, resins, BF₃ complexes, HF, H₂SO₄, AlCl₃, ionic liquids, super acids, etc.; and preferably a group VIII metal on an inorganic oxide support such as a zeolite support.

A preferred dimerization catalyst for use in the present invention is the transition metal catalyst/activating cocatalyst described in U.S. Pat. No. 6,291,733, which is herein incorporated by reference in its entirety. The process conditions described in this patent and the catalyst used are highly selective to the dimerization of alpha olefins to mostly linear internal olefin dimers. The patent states that any transition metal complex with a cocatalyst may be used as catalyst in the process. The preferred embodiment is described as utilizing an activating cocatalyst which is alumoxane or a combination of a Lewis acid and an alkylating agent. The preferred cocatalyst is modified methyl alumoxane (MMAO) used in molar excess. The preferred transition metal complexes are said to be tridentate bisimine ligands coordinated to an iron center or a combination of an iron center and aryl rings, either substituted or unsubstituted. The most preferred catalysts are catalysts 1-5 shown at column 3 of the patent.

The effective amount of the preferred catalyst of U.S. Pat. No. 6,291,733 is relatively low. With the catalyst and cocatalyst comprising less than one percent by mass of the total alpha olefin mixture, the dimerization reaction occurs in minutes. A preferred catalyst concentration is from about 0.01 to about 0.1 mg of catalyst per ml of alpha olefin monomer. A more preferred catalyst concentration is from about 0.02 to about 0.08 mg per ml of alpha olefin monomer and an even more preferred catalyst concentration is from about 0.05 to about 0.06 mg per ml of alpha olefin monomer.

Another preferred dimerization catalyst for use herein is described in U.S. Pat. No. 4,658,078, which is herein incorporated by reference in its entirety. The catalyst may comprise zirconium or a hafnium metallocene and an aluminoxane wherein the atom ratio of aluminum to the total of zirconium and/or hafnium in the catalyst ranges from about 1 to about 100. The metallocenes used may have the general formula (cyclopentadienyl)₂MY₂ wherein M is zirconium or hafnium and each Y is individually selected from the group consisting of hydrogen, C₁-C₅ alkyl, C₆-C₂₀ aryl and halogen. Preferably, Y is hydrogen, methyl, or chlorine. It is understood that the Ys may be the same or different. Included within the definition of the above cyclopentadienyl moiety is the lower alkyl (C₁-C₅)-substituted, preferably the methyl-substituted, cyclopentadienyl moiety. Specific examples of the metallocenes are dicyclopentadienyl dimethyl zirconium and bis(cyclopentadienyl) zirconium hydrogen chloride.

The isomerization reaction and the reaction involving the dimerization catalyst may take place in a batch or continuous process. These reactions may be carried out in separate reaction vessels or in the same reaction vessel. If the reactions take place in the same reaction vessel, they may take place consecutively or simultaneously.

In one embodiment of the present invention, in a batch reaction with the isomerization reaction proceeding to produce alpha olefin, the simultaneous reaction to produce longer chain internal olefins (having a higher carbon number than the feed internal olefins) from the alpha olefin may continue for a long period of time. The reaction may slow down when all of the original feed internal olefins are used up because the dimerization reaction will produce such a wide variety of dimers, including many which will not react further.

In the case where the isomerization reaction and the reaction involving the dimerization catalyst take place in the same reaction vessel, in the same reaction zone or in different reaction zones and either consecutively or simultaneously, the reaction conditions may be selected to achieve both the desired isomerization and also to achieve the desired reaction involving the dimerization catalyst. In such case, the temperature may range from about 0 to about 200° C., preferably from about 10 to about 150° C., more preferably from about 50 to about 120° C. The reaction pressure may range from about 1 to about 10,000 kPa, preferably from about atmospheric pressure to about 5000 kPa, most preferably about 100 to about 1000 kPa. Generally, these temperatures are obtained by starting the reaction at room temperature and allowing the reaction exotherm to heat the solution.

In a preferred embodiment of the present invention, the isomerization and dimerization reactions may take place in the same reaction vessel. The catalysts used may be incompatible but preferably are compatible because then the reactions may be carried out in the same zone of the reaction vessel without the necessity of keeping the catalysts separated from one another. Normally incompatible catalysts may be made compatible in the same reaction vessel by keeping them separated in different zones, for example, by way of a membrane which allows the olefin to migrate but does not allow the catalysts to contact each other. The single reaction vessel may be a fixed bed reaction vessel, an autoclave, a chemically stirred tank reactor or a catalytic distillation column reactor. More than one reactor may be used. A stacked bed reaction system is one possibility. In such a system, the top bed would have one catalyst and the lower bed would have another catalyst. This reaction may also be carried out in a series of reactors.

Alcohols derived from long chain olefins have considerable commercial importance in a variety of applications, including detergents, soaps, surfactants, freeze point depressants and lubricating oils, emollients, agricultural chemicals, and pharmaceutical chemicals. These alcohols are produced by any one of a number of commercial processes including the Oxo process and the hydroformylation of long chain olefins.

The internal olefins of this process may be converted into alcohols by the process described in U.S. Pat. No. 5,849,960, which is herein incorporated by reference in its entirety. Olefins are contacted with an isomerization catalyst to yield an isomerized olefin. This product is converted, preferably by hydroformylation, into an alcohol. In addition to the catalyst described in this patent, many other known hydroformylation catalysts may also be used to convert the internal olefins of the present invention into alcohols.

Alcohols made from the product internal olefins made by the process of this invention are suitable for the manufacture of anionic, nonionic, and cationic surfactants. The alcohols may be used as the precursor for the manufacture of anionic sulfates, including alcohol sulfates and oxyalkylated alcohol sulfates, and nonionic oxyalkylated alcohols.

These alcohols may be utilized to make detergent compositions. Detergent compositions made from linear alcohols have long been known to exhibit excellent biodegradability. In recent years, there exists a growing need to find alcohol intermediates which are both biodegradable and exhibit good detergency at cold wash temperatures. Alcohols containing some branching have become important. Such alcohols may be made from branched olefins, especially the branched internal olefins made according to the present invention.

Any technique for sulfating alcohols may be used herein. The alcohols may be directly sulfated or first oxyalkylated followed by sulfation. Sulfation and oxyalkylation processes are described in U.S. Pat. No. 5,849,960, the entire text of which is herein incorporated by reference.

The sulfated alcohols may be used as surfactants in a wide variety of applications, including granular and liquid laundry detergents, dishwashing detergents, cleaning agents, liquid soaps, shampoos, and liquid scouring agents. They are generally comprised of a number of components besides the sulfated alcohols. These components may be other surfactants, builders, cobuilders, bleaching agents and their activators, foam controlling agents, enzymes, anti-greying agents, optical brighteners, and stabilizers. It is well known in the detergent and cleaning fields which of these components are preferred for use in any particular application.

The internal olefin products of the process of the present invention can be used in oil field drilling applications as the base oil in invert drilling fluids. Internal olefin derivatives that can be made include alkyl benzene, alkyl xylene, detergent alcohols, plasticizer alcohols, alkenyl succinates, ether secondary alcohols, and diols and polyols produced by catalyzed dihydroxylation of internal olefins with the use of hydrogen peroxide.

The product internal olefins of this process may be converted into aldehydes by subjecting them to hydroformylating them with carbon monoxide and hydrogen in the presence of a hydroformylation catalyst, such as an Oxo catalyst, to form an aldehyde. Alcohols can be made from the aldehydes by judicious selection of catalysts and operating conditions.

The dimerized internal olefins may also be used to alkylate aromatic hydrocarbons to produce alkyl aromatic hydrocarbons. This process involves contacting mono-olefins with an aryl compound at alkylation conditions with an alkylation catalyst. For example, U.S. Pat. No. 6,111,158, which is herein incorporated by reference in its entirety, describes such a process wherein the catalyst is a zeolite having an NES zeolite structure type.

### EXAMPLES

Catalyst Preparation

The isomerization catalyst of Example 1 was made according to the procedure of Example I of U.S. Pat. No. 3,405,196, which is herein incorporated by reference, with certain modifications as described below. One gram of 80 mesh (0.124 apertures per square millimeter) activated alumina was introduced into a flask from which water had been removed by placing the flask under vacuum overnight before use. The flask containing the alumina was heated to 50° C. and then cooled to room temperature. All through this time it was kept under dry argon. One gram of a eutectic mixture of sodium and potassium (0.2 g Na:0.6 g K by weight) was added to the flask. The mixture was heated to 80° C. under argon for 15 minutes to melt the metal. The material in the flask changed color to a dark solid. Finally, it was cooled to room temperature.

The catalyst used in Example 2 contained sodium, potassium and silicon dioxide. It was obtained from SiGNa Chemistry, LLC, of Chemy Hill, N.J.

Reaction Feed

The internal olefin feed for both Examples 1 and 2 was a mixture of linear butenes, specifically cis-2-butene and trans-2-butene along with 15 percent by weight of 1-butene. The feed contained 99.2% of butenes with the balance being primarily butanes.

### Example 1

The internal olefin feed (10 g) and the sodium/potassium/alumina isomerization catalyst were introduced into a stirred reaction vessel at room temperature and 101 kPa, substantially in the absence of air and water. This mixture was stirred and cooled to 0° C. After about 10 minutes, the butenes were transferred into a stirred stainless steel autoclave which contained the dimerization catalyst described in Illustrative Embodiment VIII of U.S. Pat. No. 4,658,078, bis(cyclopentadienyl) zirconium hydrogen chloride (1.0 g). This mixture was allowed to react at 25° C. and atmospheric to autogenic pressure kPa for about one hour. The resulting reaction mixture contained a 1-butene depleted mixture (less than 1 wt %) of 2-butenes, octenes, and a small amount of heavier oligomers (less than 1 wt %).

Next, the dimers and unreacted butenes were cycled back to the stirred reaction vessel. The isomerization reaction in the stirred reaction vessel was allowed to continue at 70° C. and autogenic pressure kPa for about one hour at which time the reaction mixture was transferred to autoclave where further reaction with the dimerization catalyst took place at the same conditions for about one hour. After six cycles, the reaction was stopped and the reaction mixture was analyzed. The reaction mixture was cooled to 0° C. and was filtered to remove solids from the liquid product. Upon analysis, 90 percent of the feed was converted to C₈ dimers, trimers, etc. from which pure C₈ dimer was distilled at atmospheric pressure.

### Example 2

In this example, only one reaction container was utilized. The sodium/potassium/silica isomerization catalyst and the feed were introduced into the reaction container. 1 gram of the dimerization catalyst, which in this case was dicyclopentadienyl dimethyl zirconium, was added to the reaction container at 0° C. The reactions were carried out at 70° C. and kPa autogenic pressure for 4 hours. At the end of that time the reaction mixture was cooled to 0° C., filtered to remove the solids, and the liquid organic products were removed and analyzed. 87 percent had been converted to C₈ dimers, trimers, etc. from which pure C₈ dimer was distilled. Increasing the reaction time at a fixed temperature increases the ratio of trimers and heavier oligomers produced relative to the dimers. Decreasing the reaction time at a fixed temperature gives a higher ratio of dimers relative to trimers and oligomers but slows down the dimerization/oligomerization rate.

We claim:

1. A process for making internal olefin(s) which comprises isomerizing a feed comprising one or more internal olefin(s) in the presence of an isomerization catalyst to produce alpha olefin(s) and reacting said alpha olefins in the presence of a dimerization catalyst to produce internal olefin(s) which have a higher carbon number than the feed internal olefin(s).

2. The process of claim 1 wherein the amount of alpha olefins produced from the isomerization reaction is as close to the equilibrium amount of alpha olefins in the isomerization reaction mixture as possible.

3. The process of claim 1 wherein the temperature is from about 0 to about 200° C. and the pressure is from about 1 to about 10,000 kPa.

4. The process of claim 1 wherein the isomerization reaction and the reaction in the presence of the dimerization catalyst take place in the same reaction vessel.

5. The process of claim 3 wherein the temperature is from about 10 to about 150° C.

6. The process of claim 4 wherein the temperature is from about 50 to about 120° C.

7. The process of claim 6 wherein the amount of alpha olefins produced is the equilibrium amount or less of alpha olefins in the isomerization reaction mixture.

8. The process of claim 1 wherein the isomerization reaction and the reaction which takes place in the presence of the dimerization catalyst are carried out in different zones of the same reaction vessel.

9. The process of claim 1 wherein the isomerization reaction and the reaction which takes place in the presence of the dimerization catalyst are carried out in separate reaction vessels.

10. The process of claim 9 wherein the isomerization reaction takes place at a temperature of about 0 to about 500° C. and a pressure of about 1 to about 10,000 kPa.

11. The process of claim 9 wherein the dimerization reaction takes place at a temperature of up to about 200° C. and a pressure from about 1 to about 10,000 kPa.

12. The process of claim 1 wherein the isomerization and dimerization reactors take place in separate zones within the same reaction container wherein the zones permit the isomerized alpha olefins to move into the dimerization zone but prevent contact between the isomerization catalyst and dimerization catalyst.

13. The process of claim 1 wherein alpha olefins are present along with the feed internal olefins.

14. The process of claim 13 wherein the alpha olefins are selected from the group consisting of ethylene and propylene.

15. The process of claim 14 wherein the dimerized internal olefins produced have 6 or 7 carbon atoms.

16. The process of claim 1 wherein the feed internal olefins have from 4 to 24 carbon atoms and the dimerized internal olefins have from 6 to 40 carbon atoms.

17. The process of claim 16 wherein the feed internal olefins have from 4 to 20 carbon atoms and the dimerized internal olefins have from 8 to 20 carbon atoms.

18. The process of claim 17 wherein the feed internal olefins have from 4 to 14 carbon atoms and the dimerized internal olefins have from 12 to 18 carbon atoms.

19. The process of claim 1 wherein the dimerization catalyst is comprised of methyl alumoxane, tridentate bisimine ligands coordinated to an iron center or a combination of an iron center and aryl rings, either substituted or unsubstituted, and an alkylating agent.

20. The process of claim 1 wherein the dimerization catalyst comprises a metallocene having the general formula (cyclopentadienyl)₂MY₂ wherein M is zirconium or hafnium and each Y is individually selected from the group consisting of hydrogen, C₁-C₅ alkyl, C₆-C₂₀ aryl and halogen and an alumoxane wherein the atom ratio of aluminum to M in the catalyst ranges from about 1 to about 100.

21. A process for the production of alcohols which comprises first making internal olefins by the process of claim 1, contacting the internal olefins with an isomerization catalyst to yield isomerized olefins, and hydroformylating the isomerized olefins to produce alcohols.

22. A process for the production of sulfated detergents which comprises first making internal olefins by the process of claim 1, contacting the internal olefins with an isomerization catalyst to yield isomerized olefins, hydroformylating the isomerized olefins to produce alcohols, optionally oxyalkylating the alcohols, sulfating the alcohols, and combining the sulfated product with other detergent components.
